(19) United States
(12) Patent Application Publication
(10) Pub. No.: US 2007/0224300 A1
(43) Pub. Date: Sep. 27, 2007

(54) WEIGHT LOSS COMPOSITIONS USING CITRUS PEEL EXTRACT AND EURYCOMA LONGFOLIA

(76) Inventor: Shawn M. Talbott, Draper, UT (US)

Correspondence Address: HOLME ROBERTS & OWEN, LLP, 299 SOUTH MAIN, SUITE 1800, SALT LAKE CITY, UT, 84111, US

(21) Appl. No.: 11/690,754

(22) Filed: Mar. 23, 2007

Related U.S. Application Data
(60) Provisional application No. 60/785,163, filed on Mar. 23, 2006.

(51) Int. Cl. A61K 36/752; A61K 36/18; A61K 38/16; A61K 31/7048; A61K 31/353

(52) U.S. Cl. 424/736; 424/773; 514/27; 514/456; 514/2

(57) ABSTRACT

A combination of Eurycoma longifolia and citrus peel extract are provided in a supplement to promote weight loss and help dieters maintain high energy levels by reducing the body's normal shift to a catabolic state during weight loss. The Eurycoma longifolia root extract preferably includes eurypeptides and the citrus peel extract preferably includes tangeretin, nobiletin, and sinensetin. The supplement can be in any suitable form and is preferably administered in conjunction with a weight loss regime that includes a caloric deficiency diet and an exercise program.

### CROSS-REFERENCE TO RELATED APPLICATIONS

This application claims the benefit of Provisional Application No. 60/785,163, filed Mar. 23, 2006, entitled “Weight Loss Compositions Using Citrus Peel Extract And Eurycoma Longifolia,” which is incorporated by reference herein.

### BACKGROUND OF THE INVENTION

1. The Field of the Invention

The present invention relates to the fields of food compositions and dietary supplements. More particularly, the invention provides compositions and methods for the use of citrus peel extract and Eurycoma longifolia for weight loss.

2. The Relevant Technology

Obesity, diabetes, and metabolic syndrome (“Syndrome X”—encompassing insulin resistance, type 2 diabetes, dyslipidemia, hypertension, and abdominal obesity) are reaching epidemic proportions in the United States and around the world. Excess glucocorticoids such as cortisol (a primary stress hormone) have been theorized to contribute to visceral obesity and diabetes.

One way of categorizing metabolic processes, whether at the cellular, organ or organism level is as anabolic or catabolic. Catabolism is the part of metabolism that breaks down molecules into smaller units to generate energy and simultaneously takes measures to conserve energy. Stress such as weight loss and exercise can put the body into the catabolic state, in which the body can experience muscle loss, reduced metabolic rate with a corresponding reduced calorie expenditure and lower perceived energy levels, and weight gain in the form of fat. One aspect of the catabolic state is that it can be characterized as having high cortisol (a catabolic stress hormone) levels and low testosterone (an anabolic hormone) levels. Conversely, in the anabolic state the body experiences muscle maintenance or growth, normal metabolic rates, and weight loss in the form of fat. The anabolic state can be characterized by relatively low cortisol levels and high testosterone levels.

As noted above, weight loss and intense exercise both place the body in a catabolic state with reducing testosterone levels and elevating cortisol levels. While these bodily responses serve their intended role for survival, they inhibit the ability of humans to lose weight or perform at a high level during lengthy strenuous exercise. Accordingly, methods of controlling or limiting this effect would represent an advance in the art of dietary supplements.

### BRIEF SUMMARY OF THE INVENTION

The present invention uses a synergistic combination of supplements to help mammals, e.g. people, lose weight. The inventive combinations preferably include complementary does of Eurycoma longifolia and citrus peel extract to promote weight loss and help dieters maintain high energy levels by reducing the body's normal shift to a catabolic state during weight loss. The Eurycoma longifolia root extract preferably includes eurypeptides and the citrus peel extract preferably includes tangeretin, nobiletin, and sinensetin. The supplement can be in any suitable form and is preferably administered in conjunction with a weight loss regime that includes a caloric deficiency diet and an exercise program.

The current invention represents an important and innovative treatment for obesity, diabetes, and related metabolic diseases by promoting weight loss, controlling blood sugar levels, and maintaining high energy levels. The herein disclosed supplements have been found particularly effective at increasing weight loss during exercise and dieting and improving the stress levels of the subjects, which in turn increases the likelihood of a patient staying with the diet.

Accordingly, a first example embodiment of the invention is a dietary supplement including from about 1 mg to about 1000 mg of a Eurycoma longifolia root extract and from about 100 mg to about 500 mg of a citrus peel extract. The Eurycoma longifolia is preferably obtained by a hot water extraction process yielding 18-28% eurypeptides. The citrus peel extract includes polymethoxylated flavones, preferably at least one of tangeretin, nobiletin, and sinensetin. The supplement can be administered in a dietary acceptable carrier prepared in a dosage form such as, by way of example only, a shake, a beverage, liquid, a gel, a tablet, a capsule, a powder, a confectionary, or a supplemented food. Other additives that can be effectively added include, again by way of example only, whey protein, ginseng, caffeine, guarana, green tea extract, chromium, vanadium, and CLA.

Another example embodiment of the invention is a topical supplement. One example topical supplement general includes from about 1 mg to about 1000 mg of a Eurycoma longifolia root extract, from about 100 mg to about 500 mg of a citrus peel extract, each blended in a topical cream blended that is selected and configured for facilitating transdermal absorption of the citrus peel extract and Eurycoma longifolia.

Another example embodiment of the invention is a method for promoting weight loss in a human subject. The method generally includes: administering to a human subject participating in a multi-day exercise routine a dietary intake consisting of a caloric intake that is insufficient to maintain the human subject's weight over the course of the multi-day exercise routine; and administering to the human subject an oral supplement including Eurycoma longifolia and citrus peel extract, as described elsewhere herein.

Another example embodiment of the invention is another method for promoting weight loss in a human subject. This method generally includes: administering to a human subject participating in a multi-day exercise routine a dietary intake consisting of a caloric intake that is insufficient to maintain the human subject's weight over the course of the multi-day exercise routine, and administering to the human subject a topical supplement including Eurycoma longifolia and citrus peel extract, as described elsewhere herein.

Yet another example embodiment of the invention is a method for promoting weight reduction in a human subject by administering a supplement to a human subject in need thereof during weight loss. The supplement generally includes an effective dose of citrus peel extract for inhibiting weight gain or promoting weight loss in a human subject and an effective dose of Eurycoma longifolia for promoting weight loss and maintaining energy levels during weight loss in the human subject. The effective dose can be defined as the amounts sufficient to reduce salivary cortisol levels by at least about 5%, more preferably at least about 10%, still more preferably at least about 15%, all relative to cortisol levels that would result in the absence of the administration of the citrus peel extract and Eurycoma. The effective dose can also be defined as the amount sufficient to decrease the cortisol:testosterone ratio by at least about 5%, more preferably at least about 10%, still more preferably at least 15%, all relative to the cortisol:testosterone ratio that would result in the absence of the administration of the citrus peel extract and Eurycoma. This example method is preferably administered in conjunction with an exercise regime and a diet comprising a caloric deficiency.

Another example embodiment of the invention is a method for preventing or treating conditions enhanced by the presence of excess glucocorticoids, e.g. cortisol, in a mammal. This method generally includes administering to a mammal having a caloric deficiency a therapeutically effective dose of citrus peel extract and Eurycoma longifolia root extract for reducing excess glucocorticoids in the mammal. The method may optionally include placing the mammal in the caloric deficiency by administering to the mammal a reduced calorie diet in conjunction with an exercise regime. It has been found according to the invention that the therapeutically effective dose of citrus peel extract and Eurycoma longifolia root extract reduces the levels of glucocorticoids the body accumulates in response to such a caloric deficiency and exercise induced stress.

These and other objects and features of the present invention will become more fully apparent from the following description and appended claims, or may be learned by the practice of the invention as set forth hereinafter.

### DETAILED DESCRIPTION OF THE PREFERRED EMBODIMENTS

In the following description, numerous specific details are set forth in order to provide a thorough understanding of the present invention. It will be obvious, however, to one skilled in the art that the present invention may be practiced without these specific details. In other instances, well-known aspects of dietary supplements, topical creams, and the various compounds discussed herein have not been described in particular detail in order to avoid unnecessarily obscuring the present invention.

The present invention uses a synergistic combination of supplements to help mammals, e.g. people, lose weight. The inventive combinations preferably include complementary does of Eurycoma longifolia and citrus peel extract to promote weight loss and help dieters maintain high energy levels by reducing the body's normal shift to a catabolic state during weight loss. Each of these components is described generally below, followed by a discussion of the inventive compositions, with several examples.

### Citrus Peel Extract

Citrus peel extract includes Polymethoxylated Flavones (PMFs), particularly tangeretin, sinensetin, and nobilitin, which are known to have health benefits in reducing cholesterol and promoting cardiovascular health. According to the invention it has also been determined that PMFs also help reduce cortisol levels. Cortisol is a stress hormone, higher levels of which have been tied to weight gain. Accordingly, the present invention provides compositions and methods using PMFs for the prevention and treatment of diabetes and obesity. Individuals at high risk of developing or having diabetes or desiring to prevent weight gain or promote weight loss may be treated with an effective dose of citrus polmethoxylated flavones (PMFs). More particularly, the present invention relates to the discovery and use of PMFs to reduce systemic and local cortisol concentrations (liver and adipose tissue), and promoting blood sugar control and weight loss.

It has been determined that different extraction processes in fact result in different products with different concentrations and ratios of PMFs. The extraction process can therefore be selected and modified as desired to shift the ratios of the component PMFs. For example, a preferred citrus peel extract has: from about 5 mg to about 20 mg tangeretin; from about 5 mg to about 20 mg nobiletin, and from about 2 mg to about 10 mg sinensetin.

The weight loss promoting effect of citrus peel extract can be enhanced with additives such as green tea extract, chromium, vanadium, CLA, and the like.

### Eurycoma longifolia

Eurycoma (Eurycoma longifolia, also known as Tongkat ali and Malaysian ginseng) is a traditional Malaysian herbal remedy used for increasing energy levels and libido in older men. According to the invention, effective compositions and methods of using Eurycoma have now been identified for the maintenance of an anabolic hormone profile during weight loss. Individuals attempting to lose weight, for example with the use of citrus peel extract as described below, may be treated with an effective dose of Eurycoma to enhance weight loss and maintain high energy levels.

Eurycoma according to the invention helps enhance weight loss and maintain high energy levels by maintaining normal levels of cortisol and testosterone during weight loss. More particularly, Eurycoma is used to help maintain normal (low) cortisol and normal (high) testosterone levels during the stress of weight loss. This hormonal control provides energy to a person in a weight loss phase while simultaneously helping them lose weight. As a result, effective doses of Eurycoma help prevent the body from seeking to gain weight by storing fat and increasing appetite. The Eurycoma as used according to the invention can thereby help stop the “yo-yo” diet effect where a dieter's initial weight loss of a few pounds sends the body into a catabolic state, leading to binge eating and fat storage. In particular, it is believed that inhibiting or reducing the cortisol spike associated with weight loss can make it easier or a person to control their appetite. Similarly, it is believed that controlling the cortisol and testosterone levels helps prevent the body from increasing fat storage and increases a person's baseline energy.

Although not each embodiment of the invention is tied to any particular extraction process, a presently preferred extraction process is a hot water extraction process yielding 18-28% eurypeptides confirmed by HPLC analysis.

The energy enhancing effect of the Eurycoma may be increased with the aid of energy boosting supplements such as other ginseng forms, caffeine, guarana, and the like.

### Compositions

Compositions according to the invention preferably include citrus peel extract to promote weight loss and promote blood sugar control and Eurycoma to further promote weight loss while maintaining high energy levels. The use of these two compositions in conjunction with one another has been found to provide a synergistically improved dietary supplement to promote weight loss.

In addition, the foregoing components can be preferably combined with a variety of other supplements, vitamins, stabilizers, and the like as are known in the art and in view of the disclosure herein to create a desirable supplement. By way of example only, such additives can include other forms of ginseng, caffeine, guarana, green tea extract, chromium, vanadium, CLA, and the like.

According to one embodiment of the invention, a mammalian subject can be administered once a day a composition of matter having from about 10 mg to about 1,000 mg of a citrus peel extract and from about 10 mg to about 500 mg of Eurycoma root extract, preferably containing 18-28% eurypeptides. Of course, the amount of citrus peel extract and Eurycoma will preferably increase or decrease depending on the concentrations and activities of the citrus peel extract and Eurycoma root extract. In a more preferred embodiment the composition of matter has from about 25 mg to about 250 mg of a citrus peel extract and from about 25 mg to about 250 mg of Eurycoma root extract, preferably containing 18-28% eurypeptides. In a still more preferred embodiment the composition of matter has from about 50 mg to about 100 mg of a citrus peel extract and from about 50 mg to about 100 mg of Eurycoma root extract, preferably containing 18-28% eurypeptides. In various preferred embodiments the citrus peel extract preferably has at least about 10 mg PMFs and at least about 10 mg of a Eurycoma root extract containing 18-28% eurypeptides. As previously noted, the foregoing ranges of all citrus peel extract and Eurycoma components disclosed herein may increase or decrease depending on the extraction techniques and other variables.

Alternatively, in addition to or in lieu of a measured dose of citrus peel extract with the Eurycoma, the invention may include a composition of matter having one or more of tangeretin, nobiletin, and sinensetin with the Eurycoma. By way of example only, the dosage may have one or more of from about 1 mg to about 50 mg of tangeretin, from about 1 mg to about 50 mg of nobiletin, and from about 1 mg to about 20 mg of sinensetin. Of course, the dosage will increase or decrease depending on the concentration of the citrus peel extract. In a more preferred embodiment the composition of matter has from about 5 mg to about 20 mg of tangeretin, from about 5 mg to about 20 mg of nobiletin, and from about 2 mg to about 10 mg of sinensetin.

A preferred embodiment of the invention is a composition of matter having an effective dose of citrus peel extract for promoting weight loss in a mammal and an effective dose of Eurycoma longifolia for promoting weight loss and maintaining energy levels during weight loss in a mammal. As used herein, the term “an effective dose” indicates an amount sufficient to create a desirable or required result. For example, in one embodiment an effective dose is the amount necessary to obtain a measurable change in cortisol levels. One example of an effective dose may therefore an amount preferably reduce cortisol levels by at least about 5 percent, more preferably at least about 10%, still more preferably at least about 15%, all relative to a cortisol level change that would occur in the absence of taking the effective dose. In other words, an effective may be an amount that reduces cortisol gain in a dieter from 15% to 10%. Alternatively, an effective dose can be the amount necessary to promote a relative weight reduction, wherein a relative weight loss is defined by a human obtaining a lower weight than the human user would have obtained without receiving the effective dosage. The precise parameters of an effective dose may of course vary with a number of factors, including by way of example only, weight, gender, age, and desired result.

The compositions of the present invention may be formulated for administration through various known routes of administration, such as oral, parenteral, and transdermal routes. Examples of oral dosage formulations include without limitation, tablets, capsules, liquids, suspensions, gels, powders, effervescent beverages, lozenges, chewing gum, candy, supplemented food, etc. Examples of transdermal routes of administration include without limitation, topical formulations, such as lotions, creams, gels, and pastes, and transdermal patches, such as liquid reservoir patches, plasters, and adhesive matrix patches. Suitable ingredients required to produce a particular formulation, such as specific carriers, excipients, binders, penetration enhancers, etc., will be readily recognized by those of ordinary skill in the art and are not discussed in detail herein to avoid obscuring the invention.

For example, one preferred administration route is as a diet shake. The diet shake can be provided to a user in either liquid form or as a powder having suitable ingredients for the user to form a shake upon mixture with a liquid. In addition, a supplemented food such as a nutritional bar or dessert is also preferred.

The herein disclosed supplements can be preferably combined in the foregoing administration forms with a variety of other additives such as known supplements, vitamins, stabilizers, and the like as are known in the art to create a desirable administration form. By way of example only, such additives can include other forms of whey protein, ginseng, caffeine, guarana, green tea extract, chromium, vanadium, CLA, and the like.

Other conventional additives such as stabilizers, pH adjusters, excipients, carriers and diluents and the like may be added as desired or necessary as is known in the art provided they do not interfere with the activity of citrus peel extract.

The compositions of the invention can be formulated so as to provide quick, sustained or delayed release of the citrus peel extract after administration employing procedures and formulations known in the art.

For weight reduction, a regular dosage regime is preferably, though not necessarily, implemented. The generation of appropriate dosage regimes and the timing of single use applications can be determined by one of skilled in the art in view of the disclosure herein. By way of example only, however, a person in a weight loss regime can preferably take one daily supplement within the foregoing ranges of citrus peel extract and Eurycoma. Alternatively, a person can take a single supplement. The regime is preferably administered in conjunction with an exercise regime and reduced calorie diet where the citrus peel extract and Eurycoma increase weight loss and reduce stress.

The following examples are shown by way of illustration only.

### EXAMPLES

### Example 1

A weight loss shake can be prepared by adding to water a powder having an effective dose of citrus peel extract and Eurycoma longifolia in a composition as follows:

TABLE 1

| Ingredient | Amount | |
|---|---|---|
| Eurycoma longifolia | 50 | mg |
| (18–28% eurypeptides) | | |
| Citrus peel extract | 80 | mg |
| Whey protein | 20 | g |
| concentrate | | |
| Maltodextrin | 20 | g |
| Fructose | 5 | g |
| Lecithin | 2 | g |
| Total | 47.13 | g |

### Example 2

A dietary supplement for human consumption incorporating an effective dose of citrus peel extract and Eurycoma longifolia can be prepared as listed below. The supplement is preferably administered as a tablet, capsule, or powder or admixed into a food product or beverage.

TABLE 2

| Ingredient | Amount | |
|---|---|---|
| Eurycoma longifolia | 50 | mg |
| (18–28% eurypeptides) | | |
| Citrus peel extract | 80 | mg |
| Green tea | 100 | mg |
| Black tea | 100 | mg |
| Oolong tea | 100 | mg |
| Scutellaria baicalensis | 250 | mg |
| Chromium | 100 | mcg |
| Vanadium | 10 | mcg |
| 5-HTP | 100 | mg |
| Total | 780.11 | mg |

### Example 3

A twelve week study was performed to examine the effects of using a herein disclosed supplement in a combined diet/exercise/supplement regimen on weight loss and metabolic parameters. Thirty-two moderately overweight adult subjects participated in the study. They had the following characteristics:

TABLE 3

| Baseline | | | | |
|---|---|---|---|---|
| Characteristics | Age (y) | BW (kg) | BF (%) | BMI |
| N = 29 | 44 ± 8 | 84.1 ± 39.5 | 35.1% ± 9.9% | 29.5 ± 5.6 |

Subjects followed a moderate calorie-restricted diet based on resting metabolic rate (RMR), plus a moderate exercise program (5 days a week of aerobic and strength training), stress management techniques (daily), and a dietary supplement of 150 mg citrus flavenoids and 50 mg Eurycoma longifolia extract administered daily in capsule form. The study measured body weight, RMR, body fat (by BIA and skinfold), cortisol and testosterone (by salivary enzyme immunoassay), total cholesterol, LDL, HDL, glucose, and triglycerides before and after the 12-week study.

Twenty-nine subjects completed the program (9% attrition rate), suggesting that the program was easy to follow and not overly restrictive. Results shown in Table 4 below indicate mean values and indicate that the overall lifestyle program led to significant changes in body weight, body fat, testosterone, total cholesterol, and LDL (all p<0.05 by paired t-test compared to baseline values). Resting metabolic rate, cortisol, HDL, glucose, and triglycerides were unchanged.

In addition, before and after participation the subjects each completed a 65 question Profile of Mood States (POMS) questionnaire. The Profile of Mood States (POMS) is a well known test that measures present mood state (disturbance) by a list of adjectives. In the test, respondents rate 65 adjectives on a 5-point intensity scale in terms of how they have been feeling in the past week (0=not at all and 4=extremely). Except for vigor, the higher the score, the greater the mood disturbance/more distress. For analysis, the questions of the POMS are divided into six categories: tension, fatigue, confusion, vigor, depression, and anger and a score calculated for each. A Global Mood State score is also derived from the sum of all negative categories minus the score for vigor. For further details on the POMS test, see McNair D M, Lorr M, and Droppleman L F., Profile of Mood States Manual, San Diego, Calif.: Educational and Industrial Testing Services (1971). Before and after mean results are included in Table 3 below.

The data indicates that the use of the dietary supplement with Eurycoma and citrus peel extract in conjunction with a weight loss dietary regimen and aerobic and resistance exercise prevents the expected decline in fat-free mass and resting metabolic rate and results in favorable changes in body composition, metabolic hormones, mood, and cardiovascular parameters. The low attrition rate suggests that the regime was not overly stressful on users and may be expected to result in superior long-term adherence.

TABLE 4

| Measurement | Pre | Post |
|---|---|---|
| Body Weight (lbs) | 185 | 179 |
| Body Fat (%) | 35% | 33% |
| Fat Mass (lbs) | 65 | 59 |
| Fat Free Mass (lbs) | 120 | 120 |
| Resting Metabolic Rate (calories) | 1646 | 1623 |
| Cortisol-to-testosterone ratio | 0.319 | 0.272 |
| Total Cholesterol (mg/dl) | 238 | 200 |
| LDL cholesterol (mg/dl) | 149 | 121 |
| Mood (all of the following numbers are | 147 | 114 |
| scaled from a psychological survey - no | | |
| units) | | |
| Stress | 21 | 18 |
| Tension | 14 | 10 |
| Fatigue | 14 | 10 |
| Confusion | 14 | 7 |
| Vigor | 22 | 28 |
| Depression | 14 | 7 |
| Anger | 16 | 9 |

### Example 4

A six week study was performed to compare the effects of using a herein disclosed supplement in a combined diet/exercise/supplement regimen on weight loss and metabolic parameters to the effects of not using supplement in a combined diet/exercise/supplement regimen. Thirty moderately overweight adult subjects completed the study, twenty using the supplement and ten serving as controls.

The test subjects followed a moderate calorie-restricted diet based on resting metabolic rate (RMR), plus a moderate exercise program (5 days a week of aerobic and strength training), stress management techniques (daily), and a dietary supplement of 150 mg citrus flavenoids and 50 mg Eurycoma longifolia extract administered daily in capsule form. The control subjects followed the same regime but without the dietary supplement. The study measured body weight, RMR, body fat (by BIA and skinfold), cortisol and testosterone (by salivary enzyme immunoassay), total cholesterol, LDL, HDL, glucose, triglycerides, and stress factors through Profile of Mood States exam. The subjects were tested before and after the 6-week study.

Pre and post study results shown in Table 5 below for the test group indicate significant changes in mean body weight, body fat, fat mass, cortisol to testosterone ratio, total cholesterol, LDL, and MOOD (all p<0.05 by paired t-test compared to baseline values), and each of the individual mood scores from the POMS test.

TABLE 5

| Measurement | Pre | Post |
|---|---|---|
| Body Weight (lbs) | 187 | 181 |
| Body Fat (%) | 37% | 33% |
| Fat Mass (lbs) | 69 | 60 |
| Fat Free Mass (lbs) | 118 | 121 |
| Resting Metabolic Rate (calories) | 1655 | 1641 |
| Cortisol-to-testosterone ratio | .333 | .266 |
| Total Cholesterol (mg/dl) | 244 | 192 |
| LDL cholesterol (mg/dl) | 155 | 116 |
| MOOD (all of the following numbers are scaled | 152 | 112 |
| from a psychological survey - no units) | | |
| Stress | 22 | 17 |
| Tension | 16 | 10 |
| Fatigue | 16 | 7 |
| Confusion | 10 | 8 |
| Vigor | 20 | 29 |
| Depression | 15 | 7 |
| Anger | 15 | 10 |

In contrast, Results shown in Table 6 below for the control group indicate negligible changes in body weight, body fat, fat mass, total cholesterol, LDL, and MOOD (all p<0.05 by paired t-test compared to baseline values), and each of the individual mood scores. The only significant change was a dramatic jump in cortisol to testosterone ratio, suggesting that the body was under stress due to the reduced caloric intake and exercise and was increasing cortisol levels and/or decreasing testosterone levels.

TABLE 6

| Measurement | Pre | Post |
|---|---|---|
| Body Weight (lbs) | 184 | 182 |
| Body Fat (%) | 35% | 35% |
| Fat Mass (lbs) | 64 | 64 |
| Fat Free Mass (lbs) | 120 | 118 |
| Resting Metabolic Rate (calories) | 1680 | 1602 |
| Cortisol-to-testosterone ratio | .309 | .392 |
| Total Cholesterol (mg/dl) | 233 | 229 |
| LDL cholesterol (mg/dl) | 150 | 140 |
| MOOD (all of the following numbers are scaled | 144 | 144 |
| from a psychological survey - no units) | | |
| Stress | 20 | 20 |
| Tension | 15 | 14 |
| Fatigue | 15 | 13 |
| Confusion | 10 | 9 |
| Vigor | 21 | 23 |
| Depression | 14 | 11 |
| Anger | 14 | 12 |

The data indicates that the use of the dietary supplement with Eurycoma and citrus peel extract greatly improves a weight loss regiment that combined aerobic and resistance exercise program with reduced caloric intake.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A dietary supplement, comprising:
from about 1 mg to about 1000 mg of a Eurycoma longifolia root extract; and
from about 100 mg to about 500 mg of a citrus peel extract.

2. The dietary supplement of claim 1, wherein the Eurycoma longifolia is obtained by a hot water extraction process yielding 18-28% eurypeptides.

3. The dietary supplement of claim 1, wherein the effective dose comprises from about 20 mg to about 200 mg of a Eurycoma longifolia root extract containing from about 18% to about 28% eurypeptides.

4. The dietary supplement of claim 1, wherein the citrus peel extract comprises at least one of tangeretin, nobiletin, and sinensetin.

5. The dietary supplement of claim 1, wherein the citrus peel extract comprises:
from about 5 mg to about 20 mg tangeretin;
from about 5 mg to about 20 mg nobiletin, and
from about 2 mg to about 10 mg sinensetin.

6. The dietary supplement of matter of claim 1, wherein the citrus peel extract comprises at least about 10 mg polymethoxylated flavones (PMFs).

7. The dietary supplement of claim 1, wherein the citrus peel extract comprises at least about 80 mg PMFs.

8. The dietary supplement of claim 1, further comprising:
a dietary acceptable carrier prepared in a dosage form selected from the group consisting of a liquid, a gel, a tablet, a capsule, a powder, a confectionary, and a supplemented food.

9. The dietary supplement of claim 8, wherein the dietary acceptable carrier comprises an exercise beverage formulated for administration during exercise.

10. The dietary supplement of claim 8, further comprising at least one of whey protein, ginseng, caffeine, guarana, green tea extract, chromium, vanadium, and CLA.

11. A method for promoting weight loss in a human subject, the method comprising:
administering to a human subject participating in a multi-day exercise routine a dietary intake consisting of a caloric intake that is insufficient to maintain the human subject's weight over the course of the multi-day exercise routine; and
administering to the human subject a dietary supplement as defined as in claim 1.

12. A topical supplement, comprising:
from about 1 mg to about 1000 mg of a Eurycoma longifolia root extract;
from about 100 mg to about 500 mg of a citrus peel extract; and
a topical cream configured for facilitating transdermal absorption of the citrus peel extract and Eurycoma longifolia.

13. The supplement of claim 12, wherein the Eurycoma longifolia is obtained by a hot water extraction process yielding 18-28% eurypeptides.

14. A method for promoting weight loss in a human subject, the method comprising:
administering to a human subject participating in a multi-day exercise routine a dietary intake consisting of a caloric intake that is insufficient to maintain the human subject's weight over the course of the multi-day exercise routine; and
administering to the human subject a topical supplement as defined as in claim 12.

15. A method for promoting weight reduction in a human subject, the method comprising administering a supplement to a human subject in need thereof during weight loss, the supplement comprising:
an effective dose of citrus peel extract for inhibiting weight gain or promoting weight loss in a human subject; and
an effective dose of Eurycoma longifolia for promoting weight loss and maintaining energy levels during weight loss in the human subject.

16. The method of claim 15, wherein:
the effective dose of Eurycoma longifolia root extract comprises from about 1 mg to about 1000 mg; and
the effective dose of citrus peel extract comprises from about 100 mg to about 500.

17. The dietary supplement of claim 15, wherein the effective doses are defined as sufficient to reduce salivary cortisol levels by at least 5% relative to cortisol levels that would result in the absence of the administration of the citrus peel extract and Eurycoma.

18. The dietary supplement of claim 15, wherein the effective doses are defined as sufficient to decrease the cortisol:testosterone ratio by at least 5% relative to the cortisol:testosterone ratio that would result in the absence of the administration of the citrus peel extract and Eurycoma.

19. The method of claim 15, further comprising administering the supplement daily in conjunction with an exercise regime and a diet comprising a caloric deficiency.

20. The method of claim 15, wherein the Eurycoma longifolia root extract comprises from about from about 18% to about 28% eurypeptides.

21. The method of claim 15, wherein the Eurycoma longifolia root extract comprises from about 20 mg to about 200 mg of a Eurycoma longifolia root extract containing from about 18% to about 28% eurypeptides.

22. The method of claim 15, wherein the supplement is in a oral form selected from the group consisting of a shake, beverage, a tablet, a capsule, a powder, a confectionary, and a supplemented food.

23. The method of claim 15, wherein the supplement is blended with a topical cream for topical administration and the act of administering the supplement comprises topically administering the supplement.

24. The method of claim 15, wherein the citrus peel extract comprises at least one of tangeretin, nobiletin, and sinensetin.

25. A method for preventing or treating conditions enhanced by the presence of excess glucocorticoids in a mammal, the method comprising:
administering to a mammal having a caloric deficiency a therapeutically effective dose of citrus peel extract and Eurycoma longifolia root extract for reducing excess glucocorticoids in the mammal.

26. The method of claim 25, wherein the therapeutically effective dose of citrus peel extract comprises from about 100 mg to about 500 mg and the therapeutically effective dose of Eurycoma longifolia root extract comprises from about 1 mg to about 1000 mg.

27. The method of claim 26, wherein the Eurycoma longifolia root extract comprises from about from about 18% to about 28% eurypeptides.

28. The method of claim 26, further comprising placing the mammal in said caloric deficiency by administering to the mammal a reduce calorie diet in conjunction with an exercise regime.

29. The method of claim 25, wherein the excess glucocorticoids are cortisol.

30. A method for promoting weight reduction in a human subject, the method comprising administering a supplement to a human subject in need thereof during weight loss, the supplement comprising:
from about 1 mg to about 300 mg eurypeptides; and
at least about 10 mg polymethoxylated flavones.
